(19) United States
(12) Patent Application Publication
(10) Pub. No.: US 2022/0232881 A1
(43) Pub. Date: Jul. 28, 2022

(54) METHOD FOR SEALING POUCHES

(71) Applicant: NICOVENTURES TRADING LIMITED, London (GB)

(72) Inventors: David Neil McClanahan, Winston-Salem, NC (US); Travis O'Neal, Pinnacle, NC (US); Bridget B. Crump, Greensboro, NC (US)

(21) Appl. No.: 17/584,476

(22) Filed: Jan. 26, 2022

Related U.S. Application Data
(60) Provisional application No. 63/142,838, filed on Jan. 28, 2021.

(51) Int. Cl. A24B 13/00; B29C 65/04; B29C 65/00; A61K 9/00

(52) U.S. Cl. CPC A24B 13/00; B29C 65/04; B29L 2031/7128; B29C 66/7294; A61K 9/009; B29C 66/433

(57) ABSTRACT

Alternative methods for sealing pouch materials, for example, using radio frequency (RF) sealing techniques, and oral pouched products formed according to those methods are provided. Some pouched products may include an outer water-permeable pouch defining a cavity having a composition situated in the cavity, wherein the outer water-permeable pouch comprises a fleece material, the fleece material comprising a plurality of fibers and an RF sealable material. In some embodiments, methods of RF sealing pouch materials may include providing one or more fleece materials having a RF sealable material and sealing the one or more fleece materials using radio frequency energy to form a RF sealed pouched product.

### CROSS-REFERENCE TO RELATED APPLICATIONS

The present application claims priority to and the benefit of U.S. Provisional Application No. 63/142,838, filed Jan. 28, 2021, the disclosure of which is incorporated by reference herein in its entirety.

### FIELD OF THE DISCLOSURE

The present disclosure relates to products intended for human use. The products are configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Certain types of pouches or sachets have been employed to contain compositions adapted for oral use. See for example, the types of representative smokeless tobacco products, as well as the various smokeless tobacco formulations, ingredients and processing methodologies, referenced in the background art set forth in US Pat. Pub. Nos. 2011/0303511 to Brinkley et al. and 2013/0206150 to Duggins et al.; which are incorporated herein by reference. During use, those pouches or sachets are inserted into the mouth of the user, and water soluble components contained within those pouches or sachets are released as a result of interaction with saliva.

Certain commercially available smokeless tobacco products, such as products commonly referred to as “snus,” comprise ground tobacco materials incorporated within sealed pouches. Representative types of snus products have been manufactured in Europe, particularly in Sweden, by or through companies such as Swedish Match AB (e.g., for brands such as General, Ettan, Goteborgs Rape and Grovsnus); Fiedler & Lundgren AB (e.g., for brands such as Lucky Strike, Granit, Krekt and Mocca); JTI Sweden AB (e.g., for brands such as Gustavus) and Rocker Production AB (e.g., for brands such as Rocker). Other types of snus products have been commercially available in the U.S.A. through companies such as Philip Morris USA, Inc. (e.g., for brands such as Marlboro Snus); U.S. Smokeless Tobacco Company (e.g., for brands such as SKOAL Snus) and R. J. Reynolds Tobacco Company (e.g., for brands such as CAMEL Snus). See also, for example, Bryzgalov et al., 1N1800 Life Cycle Assessment, Comparative Life Cycle Assessment of General Loose and Portion Snus (2005); which is incorporated herein by reference.

Various types of snus products, as well as components for those products and methods for processing components associated with those products, have been proposed. See, for example, U.S. Pat. No. 8,067,046 to Schleef et al. and U.S. Pat. No. 7,861,728 to Holton, Jr. et al.; US Pat. Pub. Nos. 2004/0118422 to Lundin et al.; 2008/0202536 to Torrence et al.; 2009/0025738 to Mua et al.; 2011/0180087 to Gee et al.; 2010/0218779 to Zhuang et al.; 2010/0294291 to Robinson et al.; 2010/0300465 to Zimmermann; 2011/0061666 to Dube et al.; 2011/0303232 to Williams et al.; 2012/0067362 to Mola et al.; 2012/0085360 to Kawata et al.; 2012/0103353 to Sebastian et al. and 2012/0247492 to Kobal et al.; and PCT Pub. Nos. WO 05/063060 to Atchley et al. and WO 08/56135 to Onno; which are incorporated herein by reference. In addition, certain quality standards associated with some snus manufacturing processes have been assembled as a so-called GothiaTek® standard. Furthermore, various manners and methods useful for the production of snus types of products have been proposed. See, for example, U.S. Pat. No. 4,607,479 to Linden and U.S. Pat. No. 4,631,899 to Nielsen; and US Pat. Appl. Pub. Nos. 2008/0156338 to Winterson et al.; 2010/0018539 to Brinkley et al.; 2010/0059069 to Boldrini; 2010/0071711 to Boldrini; 2010/0101189 to Boldrini; 2010/0101588 to Boldrini; 2010/0199601 to Boldrini; 2010/0200005 to Fallon; 2010/0252056 to Gruss et al.; 2011/0284016 to Gunter et al.; 2011/0239591 to Gruss et al.; 2011/0303511 to Brinkley et al.; 2012/0055493 to Novak III et al. and 2012/0103349 to Hansson et al.; and PCT Pub. Nos. WO 2008/081341 to Winterson et al. and WO 2008/146160 to Cecil et al.; which are incorporated herein by reference. Additionally, snus products can be manufactured using equipment such as that available as SB 51-1/T, SBL 50 and SB 53-2/T packaging machines from Merz Verpackungmaschinen GmBH.

Certain types of products employing pouches or sachets that contain tobacco substitutes (or combinations of tobacco and tobacco substitutes) also have been proposed. See, for example, U.S. Pat. No. 5,167,244 to Kjerstad and U.S. Pat. No. 7,950,399 to Winterson et al.; and US Pat. Appl. Pub. Nos. 2005/0061339 to Hansson et al.; 2011/0041860 to Essen et al. and 2011/0247640 to Beeson et al.; which are incorporated herein by reference.

Certain types of product employing pouches or sachets have been employed to contain nicotine, such as those used for nicotine replacement therapy (NRT) types of products (e.g., a pharmaceutical product distributed under the tradename ZONNIC® by Niconovum AB). See also, for example, the types of pouch materials and nicotine-containing formulations set forth in U.S. Pat. No. 4,907,605 to Ray et al.; US Pat. Appl. Pub. Nos. 2009/0293895 to Axelsson et al. and 2011/0268809 to Brinkley et al.; and PCT Pub. Nos. WO 2010/031552 to Axelsson et al. and WO 2012/134380 to Nilsson; which are incorporated herein by reference.

All-white snus portions are growing in popularity, and offer a discrete and aesthetically pleasing alternative to traditional snus. Such modern “white” pouched products may include a bleached tobacco or may be tobacco-free.

To manufacture pouched products of various types as noted above, the pouches must be sealed after being filled with the desired material. As noted in US Pat. Pub. No. 2014/0026912 to Rushforth et al., such sealing is typically accomplished by application of a binder material to the fiber network from which the pouch is constructed, which enables the pouch to be sealed upon application of heat. However, conventional binders applied to such fibrous pouches, such as acrylic polymers, are costly to apply to pouches and inhibit biodegradability of the discarded pouch. It would be useful to provide alternative methods for sealing pouched products.

### BRIEF SUMMARY

The present disclosure generally provides oral pouched products, including, but not limited to all-white snus portions. The products may be configured to impart a taste when used orally and, additionally or alternatively, may deliver active ingredients to a consumer, such as nicotine. The products and methods of the present disclosure in particular relate to alternative methods of sealing fleece materials and oral pouched products formed therefrom.

In one aspect, the present disclosure provides a pouched product including an outer water-permeable pouch defining a cavity and a composition situated in the cavity, wherein the outer water-permeable pouch includes a fleece material, the fleece material having a plurality of fibers and an RF sealable material. In some embodiments, the RF sealable material is a polar polymer material. In some embodiments, the RF sealable material is selected from the group consisting of acrylonitrile butadiene styrene (ABS) resins or polymers, acrylonitrile-methyl acrylate copolymer (AMAC), butyrate, cellulose acetate, cellulose acetate butyrate, cellulose nitrate, cellulose triacetate, various epoxy resins, ethylene-vinyl acetate (EVA), ethyl vinyl alcohol (EVOH), melamine-formaldehyde resin, methyl acrylate, pelathane, polyethylene terephthalate (PET), polyethylene terephthalate glycol-modified (PET-G), polyvinyl acetate (PVA), polyvinylchloride (PVC), polyvinylidene chloride, polyurethane, polyolefin, nylon, thermoplastic polyurethanes, open celled polyurethanes, low-density polyethylene (LDPE), and combinations thereof. In some embodiments, the RF sealable material is in the form of a plurality of RF sealable fibers, a liquid coating, a spray coating, a powder, or any combination thereof.

In some embodiments, the pouched product may include a sealed seam or multiple sealed seams (e.g., such as two- or three- or four-sealed seams). In some embodiments, the sealed seam(s) may be sealed via application of radio frequency energy. In some embodiments, the sealed seam(s) may have a width of less than about 2 mm. In some embodiments, the composition within the cavity of the pouch may include a particulate tobacco material, nicotine, particulate non-tobacco material treated to contain nicotine and/or flavoring agents, fibrous plant material carrying a tobacco extract, and combinations thereof. In some embodiments, the particulate tobacco material is in the form of a whitened tobacco material. In some embodiments, the composition is substantially free of a tobacco material. In some embodiments, the composition may include an active ingredient selected from the group consisting of a nicotine component, botanicals, stimulants, medicinals, nutraceuticals, amino acids, vitamins, cannabinoids, and combinations thereof. In some embodiments, the composition may include one or more additives selected from the group consisting of a salt, a sweetener, a binding agent, water, a humectant, a gum, an organic acid, a buffering agent, a tobacco derived material, and combinations thereof.

Some aspects of the present disclosure provide methods of RF sealing pouch materials. For instance, a method of RF sealing pouch materials may include providing one or more fleece materials having an RF sealable material and sealing the one or more fleece materials along a seam using radio frequency energy to form an RF sealed pouch material. In some embodiments, the step of sealing the one or more fleece materials may include application of radio frequency energy to the seam in the range of about 1 MHz to about 100 MHz. In some embodiments, the radio frequency energy is applied via an RF sealing die. In some embodiments, the RF sealing die is in the form of two or more electrodes configured to emit radio frequency energy.

In some embodiments, methods as described herein may include applying pressure to the one or more fleece materials during or after the sealing step. In some embodiments, the amount of pressure applied to the one or more fleece materials may be in the range of about 20 psi to about 200 psi. In some embodiments, applying pressure may include applying pressure via mechanical press, hydraulic press, pneumatic press, or any combination thereof. In some embodiments, the method may include cooling the pouch material after sealing. In some embodiments, the RF sealed pouch material may include a sealed seam or multiple sealed seams. In such embodiments, the sealed seam(s) may have a width of less than about 2 mm.

Some aspects of the present disclosure provide methods of preparing RF sealed pouched products. For instance, such methods may include providing an outer-water permeable pouch including an RF sealable material, the outer water-permeable pouch defining a cavity with a composition situated therein; and sealing a leading and an end edge of an outer water-permeable pouch using radio frequency energy to form an RF sealed pouched product. In some embodiments, methods as described herein may include applying pressure to the outer water-permeable pouch. In some embodiments, the methods may include cooling the sealed pouched product after sealing. In some embodiments, such methods may include providing a continuous supply of a fleece material having a plurality of fibers and an RF sealable material, engaging lateral edges of the fleece material such that a longitudinally-extending seam is formed, sealing the longitudinally-extending seam such that a continuous tubular member is formed from the continuous supply of fleece material, inserting a composition adapted for oral use into the continuous tubular member, and subdividing the continuous tubular member into discrete pouch portions such that each pouch portion includes a composition charge.

A further aspect of the present disclosure provides pouched products prepared according to any of the methods disclosed herein. In some embodiments, the sealed leading edge and the sealed end edge of those pouched products may both have a width of less than about 2 mm. In some embodiments, the pouched product exhibits enhanced organoleptic properties as compared to a pouched product that has not been sealed using radio frequency energy.

The disclosure includes, without limitations, the following embodiments.

Embodiment 1: A pouched product comprising: an outer water-permeable pouch defining a cavity; and a composition situated in the cavity; wherein the outer water-permeable pouch comprises a fleece material, the fleece material comprising a plurality of fibers and an RF sealable material.
Embodiment 2: The pouched product according to embodiment 1, wherein the RF sealable material is a polar polymer material.
Embodiment 3: The pouched product according to any of embodiments 1-2, wherein the RF sealable material is selected from the group consisting of acrylonitrile butadiene styrene (ABS) resins or polymers, acrylonitrile-methyl acrylate copolymer (AMAC), butyrate, cellulose acetate, cellulose acetate butyrate, cellulose nitrate, cellulose triacetate, various epoxy resins, ethylene-vinyl acetate (EVA), ethyl vinyl alcohol (EVOH), melamine-formaldehyde resin, methyl acrylate, pelathane, polyethylene terephthalate (PET), polyethylene terephthalate glycol-modified (PET-G), polyvinyl acetate (PVA), polyvinylchloride (PVC), polyvinylidene chloride, polyurethane, polyolefin, nylon, thermoplastic polyurethanes, open celled polyurethanes, low-density polyethylene (LDPE), and combinations thereof.
Embodiment 4: The pouched product according to any of embodiments 1-3, wherein the RF sealable material is in the form of a plurality of RF sealable fibers, a liquid coating, a spray coating, a powder, or any combination thereof.
Embodiment 5: The pouched product according to any of embodiments 1-4, further comprising at least two sealed seams at opposing ends of the pouched product.
Embodiment 6: The pouched product according to any of embodiments 1-5, wherein the at least two sealed seams have been sealed via application of radio frequency energy.
Embodiment 7: The pouched product according to any of embodiments 1-6, wherein the at least two sealed seams have a width of less than about 2 mm.
Embodiment 8: The pouched product according to any of embodiments 1-7, wherein the composition within the cavity of the pouch comprises a particulate tobacco material, nicotine, a particulate non-tobacco material treated to contain nicotine and/or flavoring agents, fibrous plant material carrying a tobacco extract, and combinations thereof.
Embodiment 9: The pouched product according to any of embodiments 1-8, wherein the particulate tobacco material is in the form of a whitened tobacco material.
Embodiment 10: The pouched product according to any of embodiments 1-9, wherein the composition is substantially free of a tobacco material.
Embodiment 11: The pouched product according to any of embodiments 1-10, wherein the composition comprises an active ingredient selected from the group consisting of a nicotine component, botanicals, stimulants, medicinals, nutraceuticals, amino acids, vitamins, cannabinoids, and combinations thereof.
Embodiment 12: The pouched product according to any of embodiments 1-11, wherein the composition comprises one or more additives selected from the group consisting of a salt, a sweetener, a binding agent, water, a humectant, a gum, an organic acid, a buffering agent, a tobacco derived material, and combinations thereof.
Embodiment 13: A method of RF sealing pouch materials, comprising: providing one or more fleece materials comprising an RF sealable material; sealing the one or more fleece materials along a seam using radio frequency energy to form an RF sealed pouch material.
Embodiment 14: The method according to embodiment 13, wherein the step of sealing the one or more fleece materials comprises application of radio frequency energy to the seam in the range of about 1 MHz to about 100 MHz.
Embodiment 15: The method according to any of embodiments 13-14, wherein the radio frequency energy is applied via an RF sealing die.
Embodiment 16: The method according to any of embodiments 13-15, wherein the RF sealing die is in the form of two or more electrodes configured to emit radio frequency energy.
Embodiment 17: The method according to any of embodiments 13-16, further comprising applying pressure to the one or more fleece materials during or after the sealing step.
Embodiment 18: The method according to any of embodiments 13-17, wherein the amount of pressure applied to is in the range of about 20 psi to about 200 psi.
Embodiment 19: The method according to any of embodiments 13-18, wherein the pressure is applied via mechanical press, hydraulic press, pneumatic press, or any combination thereof.
Embodiment 20: The method according to any of embodiments 13-19, further comprising cooling the pouch material after the sealing step.
Embodiment 21: The method according to any of embodiments 13-20, wherein the RF sealed pouch material comprises at least two sealed seams.
Embodiment 22: The method according to any of embodiments 13-21, wherein the at least two sealed seams have a width of less than about 2 mm.
Embodiment 23: A method of preparing an RF sealed pouched product, the method comprising: providing an outer-water permeable pouch comprising an RF sealable material, the outer water-permeable pouch defining a cavity with a composition situated therein; and sealing a leading and an end edge of an outer water-permeable pouch using radio frequency energy to form an RF sealed pouched product.
Embodiment 24: The method according to embodiment 23, further comprising applying pressure to the outer water-permeable pouch during or after the sealing step.
Embodiment 25: The method according to any of embodiments 23-24, further comprising cooling the sealed leading edge and the sealed end edge after the sealing step.
Embodiment 26: The method according to any of embodiments 23-25, further comprising: providing a continuous supply of a fleece material comprising a plurality of fibers and an RF sealable material; engaging lateral edges of the fleece material such that a longitudinally-extending seam is formed; sealing the longitudinally-extending seam such that a continuous tubular member is formed from the continuous supply of fleece material; inserting a composition adapted for oral use into the continuous tubular member; and subdividing the continuous tubular member into discrete pouch portions such that each pouch portion includes a composition charge.
Embodiment 27: A pouched product prepared according to the method of embodiment 23.
Embodiment 28: The pouched product according to embodiment 27, wherein the sealed leading edge and the sealed end edge both have a width of less than about 2 mm.
Embodiment 29: The pouched product according to any of embodiments 27-28, wherein the pouched product exhibits enhanced organoleptic properties as compared to a pouched product that has not been sealed using radio frequency energy.
Embodiment 30: The pouched product according to any of embodiments 27-29, wherein the enhanced organoleptic properties are selected from the group consisting of texture, mouthfeel, softness, stiffness, firmness, hardness, stickiness, fluffiness, durability, chewability, workability, tackiness, and combinations thereof.

These and other features, aspects, and advantages of the disclosure will be apparent from a reading of the following detailed description together with the accompanying drawings, which are briefly described below. The invention further embodiments beyond the above-noted embodiments, including any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are according to example embodiments only, and should not be construed as limiting the disclosure.

FIG. 1 is a front perspective view illustrating a pouched product according to an example embodiment of the present disclosure;

FIG. 2A is a two-dimensional view of a rectangular shaped pouch product that has been sealed along the perimeter of the pouched product and a cut-away view depicting a width of two sealed seams therein, according to an example embodiment of the present disclosure;

FIG. 2B is a two-dimensional view of a substantially circular shaped pouch product that has been sealed along the perimeter of the pouched product and a cut-away view depicting a width of a curved sealed seam therein, according to an example embodiment of the present disclosure;

FIG. 2C is an example of a pouched product according to an example embodiment of the present disclosure;

FIG. 2D is an example of a pouched product according to an example embodiment of the present disclosure; and

FIG. 3 is a flow chart illustrating the general steps for manufacturing RF sealed pouch products that have been sealed using radio frequency energy, according to an example embodiment of the present disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the mixture including water. Unless otherwise indicated, reference to “weight percent” of a mixture reflects the total wet weight of the mixture (i.e., including water).

The disclosure generally provides products configured for oral use. The term “configured for oral use” as used herein means that the product is provided in a form such that during use, saliva in the mouth of the user causes one or more of the components of the mixture (e.g., flavoring agents and/or nicotine) to pass into the mouth of the user. In certain embodiments, the product is adapted to deliver components to a user through mucous membranes in the user's mouth and, in some instances, said component is an active ingredient (including, but not limited to, for example, nicotine) that can be absorbed through the mucous membranes in the mouth when the product is used.

Some aspects of the present disclosure provide pouched products and methods of forming those pouched products. The products described herein may comprise fleece materials that are in the form of a water-permeable pouch material that surrounds a composition/mixture, also referred to herein as a “material” (e.g., a composition comprising one or more active ingredients and one or more additional components), and such pouched products may be adapted to or configured to provide for release of the one or more components within the material, such as when in contact with the oral cavity of the user of the product. The composition positioned within the pouch can be any composition containing a water-soluble component capable of being released through the water-permeable pouch, such as tea or coffee materials (e.g., in the context of a beverage pouch adapted for brewing or steeping) or compositions adapted for oral use (e.g., tobacco-derived products such as snus or nicotine replacement therapy products). In certain embodiments, the composition within the cavity of the pouch can comprise a particulate tobacco material, nicotine, particulate non-tobacco material (e.g., microcrystalline cellulose, or “MCC”) that has been treated to contain nicotine and/or flavors, fibrous plant material (e.g., beet root fiber) treated to contain a tobacco extract, and/or combinations thereof.

Such compositions in the water-permeable pouch format are typically used by placing a pouch containing the composition in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the composition therein (e.g., flavoring agents and/or nicotine) to pass through e.g., the water-permeable pouch and provide the user with flavor and satisfaction, and the user is not required to spit out any portion of the mixture. After about 10 minutes to about 60 minutes, typically about 15 minutes to about 45 minutes, of use/enjoyment, substantial amounts of the mixture have been ingested by the human subject, and the pouch may be removed from the mouth of the consumer for disposal. Preferred pouch materials for products described herein may be designed and manufactured such that under conditions of normal use, a significant amount of the contents of the formulation within the pouch permeate through the pouch material prior to the time that the pouch undergoes loss of its physical integrity.

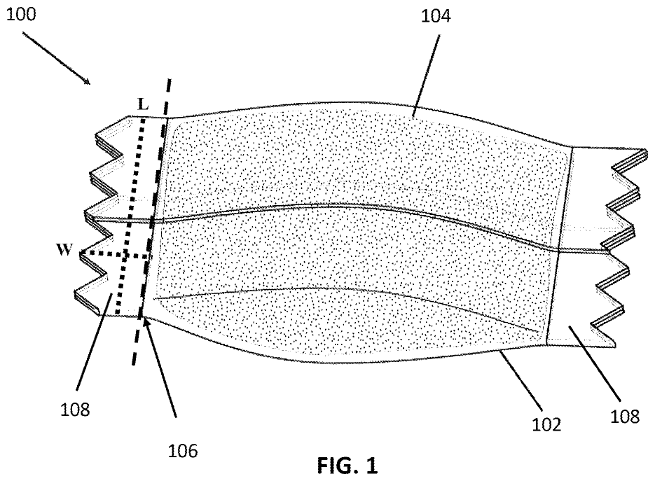

Some aspects of the present disclosure provide for pouched products that have been sealed using radio frequency energy (“RF sealed” pouched products) and various methods of sealing pouched products using radio frequency energy. For example, depicted in FIG. 1 is a pouched product 100 according to an example embodiment disclosed herein below that has been RF sealed. The pouched product 100 includes an outer water permeable pouch 102 defining a cavity, and a composition 104 situated within the cavity, wherein the outer water permeable pouch 102 comprises a fleece material, the fleece material comprising a plurality of fibers and an RF sealable material. In some embodiments, the composition within the cavity may comprise a mixture of various different components.

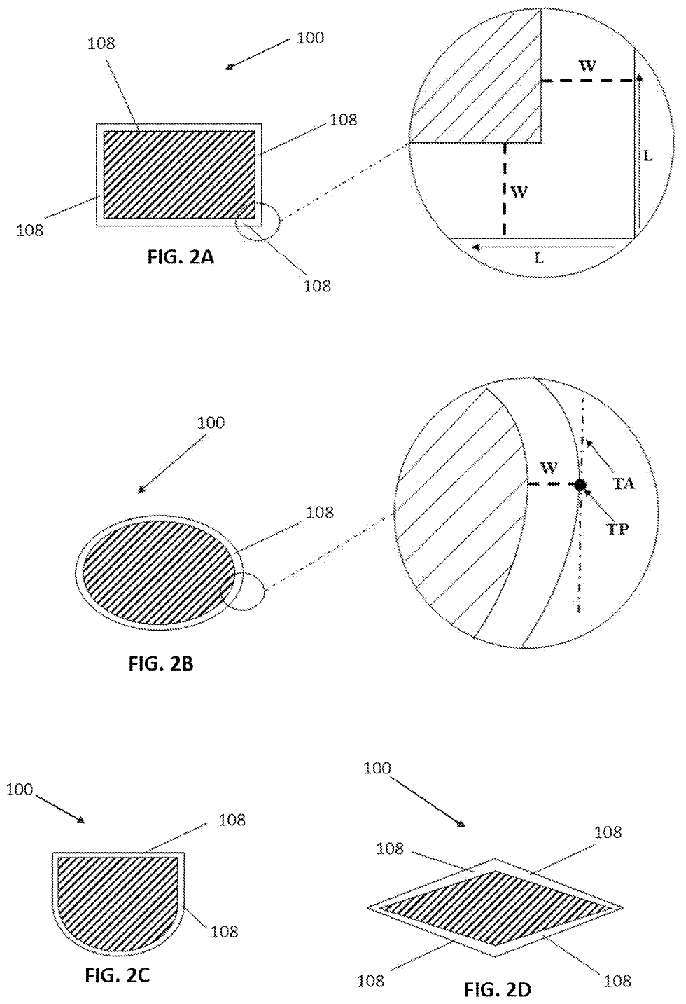

In some embodiments, the fleece material may refer to a single fleece material (e.g., when the fleece material has been sealed along a longitudinally-extending seam to form a tubular member enclosing the composition), or two or more fleece materials (e.g., layered on top of each other with a composition layer in between), that have been sealed along a linear axis (e.g., 106 in FIG. 1) to form two sealed seams 108 (e.g., generally in the form of flaps extending from the linear axis) at opposing ends of the pouched product 100. Generally, the sealed seams 108 as described herein may be defined as having both a length L (e.g., parallel to axis 106 in the embodiment depicted in FIG. 1) and a width W (e.g., perpendicular to axis 106 in the embodiment depicted in FIG. 2). In some embodiments, these seams may be substantially rectangular in shape, or in other embodiments they may have one or more cutouts as depicted in FIG. 1; however, the overall shape of the seam is not meant to be limiting. For example, the “width (W)” of a seam as defined herein refers to the largest width measured at any position along the length L of the seam 108. It should be noted that the length of the sealed seams in traditional pouched products may vary based on the size of the overall pouched product (e.g., the length of the seam is generally equal to the length of the pouched product measured at any point along axis 106), whereas the width of the seams traditionally remains constant, regardless of the size of the pouched product, because the width of the sealed seam is determined by the typical heat sealing processes used to seal these seams during production (e.g., a certain seam width, typically about 4 mm or more, is required to ensure a sufficient seal using traditional heat sealing processes).

In some embodiments, the pouched product may comprise multiple sealed seams (e.g., including two-, three-, four-, or more-seam configurations as discussed herein below). For example, in some embodiments the entire perimeter of the outer water-permeable pouch may be completely sealed using the sealing processes described herein. FIG. 2A depicts an embodiment of a pouched product 100 where the entire perimeter of the outer water-permeable pouch may be completely sealed using the sealing processes described herein, thus defining a pouched product having four separate seams 108. As shown in the cut-away view in FIG. 2A, each individual seam may have a defined width (W) along the length (L) of the seam 108 and which is measured perpendicular to the length (L) of the seam.

The disclosure provides, in additional embodiments, pouched products of shapes other than conventional rectangles and squares (as referenced herein above with respect to “conventional” pouched products). Such products are provided in varying shapes and sizes and may include any number of individual sealed seams thereon. The exact shapes of pouches are not particularly limited. In certain preferred embodiments, shaped pouches provided herein comprise at least one rounded dimension/edge. Various shapes can be described, for example, as “circular,” “oval,” “oblong,” “crescent-shaped,” “rounded crescent-shaped,” “half-moon-shaped,” “half-circular,” “teardrop-like,” “star-shaped,” “domed,” “rhombic,” “rounded rhombic,” “diamond-shaped,” “rounded diamond-shaped,” “kidney-shaped,” “heart-shaped,” “triangular,” “rounded triangular” (including, e.g., isosceles, equilateral, scalene, acute, right, and obtuse) “hexagonal,” “rounded hexagonal” (including hexagonal with equal length edges and with varying length edges) and the like. FIG. 2B, for example, depicts an embodiment of a pouched product 100 having a substantially circular shape, such that a single sealed seam 108 is form around the circumference of the outer water permeable pouch. In the depicted embodiment, and other possible embodiments, one or more of the sealed seams may not have a linear profile (e.g., the seam itself may be substantially curved, e.g., in an arc shape). In such embodiments, the width (W) of an individual seam may be defined relative to a tangential point (TP) along the perimeter of the curved seam. Thus, the width (W) of a curved seam may be defined as the largest width measured along any tangential point (TP) along the perimeter of the seam 108 and which is measured perpendicular to the tangential axis (TA) of said tangential point.

As will be discussed below in more detail, in some embodiments, the sealed seams of the pouched products provided herein may be sealed via application of radio frequency energy. Advantageously, all such seams may be sealed in accordance with the disclosed radio frequency-based methods provided herein. It should be noted that, in some embodiments, radio frequency sealed seams can advantageously provide for a seam width that is smaller than conventionally sealed pouch products (e.g., using traditional heat-sealing techniques). In some embodiments, for example, the sealed seams, sealed via radio frequency energy, may each have a width (W, as depicted in FIGS. 1 and 2A-2D) of less than about 4 mm, less than about 3 mm, less than about 2 mm, less than about 1 mm, or less than about 0.5 mm. In some embodiments, the width of each of the at least two seams may be in the range of about 0.5 mm to about 2.5 mm, about 0.75 mm to about 2 mm, or about 1 mm to about 1.5 mm. Without intending to be bound by theory, it should be noted that providing pouched products with smaller sealed seams (e.g., by using radio frequency energy to seal the seams) can advantageously provide a pouched product with enhanced organoleptic properties (e.g., texture, mouthfeel, etc.) and optionally may also reduce the amount of fleece material used for the individual pouched products. The enhanced organoleptic properties associated with the disclosed pouched products may include, but are not limited to, softness, stiffness, firmness, hardness, stickiness, fluffiness, durability, chewability, workability, tackiness, and the like. Various types of fleece materials, RF sealable materials, and methods of forming RF sealable pouched products therefrom will be discussed in further detail below.

In addition to size of the individual seams, the sizes of the shaped pouched products provided herein can vary widely. In some embodiments, the shaped pouched products may be designed so as to be substantially similar in size to conventional pouched products. In other embodiments, they may be somewhat larger in size or somewhat smaller in size.

### Fleece Materials

As referenced above, the pouched products provided herein comprise at least one fleece material. “Fleece materials” as referred to herein may be in the form of a fleece fabric material, such as in the form of a woven or nonwoven fabric comprising a plurality of fibers.

As used herein, the term “fiber” is defined as a basic element of textiles. Fibers are often in the form of a rope- or string-like element. As used herein, the term “fiber” is intended to include fibers, filaments, continuous filaments, staple fibers, and the like. In some embodiments, the fleece materials described herein may comprise multicomponent fibers. The term “multicomponent fibers” refers to fibers that comprise two or more components that are different by physical or chemical nature, including bicomponent fibers. Specifically, the term “multicomponent fibers” includes staple and continuous fibers prepared from two or more polymers present in discrete structured domains in the fiber, as opposed to blends where the domains tend to be dispersed, random or unstructured.

The term “nonwoven” is used herein in reference to fibrous materials, webs, mats, batts, or sheets in which fibers are aligned in an undefined or random orientation. The nonwoven fibers are initially presented as unbound fibers or filaments. An important step in the manufacturing of nonwovens involves binding the various fibers or filaments together. The manner in which the fibers or filaments are bound can vary, and include thermal, mechanical and chemical techniques that are selected in part based on the desired characteristics of the final product, as discussed in more detail herein below.

In some embodiments, fleece materials of the present disclosure may be formed from various types of fibers (e.g., conventional cellulosic fibers (e.g., such as viscose fibers, regenerated cellulose fibers, cellulose fibers, and wood pulps), cotton fibers, wool fibers, other natural fibers, polymer/synthetic-type fibers, and combinations thereof) capable of being formed into a traditional fleece fabrics or other traditional pouch materials. For example, fleece materials may be provided in the form of a woven or nonwoven fabric. Suitable types of fleece materials, for example, are described in U.S. Pat. No. 8,931,493 to Sebastian et al.; US Pat. Appl. Pub. Nos. 2016/0000140 to Sebastian et al. and 2016/0073689 to Sebastian et al.; which are all incorporated herein by reference. In some embodiments, the fibers within the fleece material may include, but are not limited to, a polymer selected from the group consisting of polyglycolic acid, polylactic acid, polyhydroxyalkanoates, polycaprolactone, polybutylene succinate, polybutylene succinate adipate, and copolymers thereof. In some embodiments, the fibers within the fleece material may be selected from the groups consisting of cellulose fibers, viscose fibers, regenerated cellulose fibers, other wood fibers, and the like.

Nonwoven fabric forming methods for natural and synthetic fibers may include drylaid, airlaid and wetlaid methods. In some embodiments, the nonwoven fabric can be formed using a spunlaid or spunmelt process, which includes both spunbond and meltblown processes, wherein such processes are understood to typically entail melting, extruding, collecting and bonding thermoplastic polymer materials to form a fibrous nonwoven web. The technique of meltblowing is known in the art and is discussed in various patents, for example, U.S. Pat. No. 3,849,241 to Butin, U.S. Pat. No. 3,987,185 to Buntin et al., U.S. Pat. No. 3,972,759 to Buntin, and U.S. Pat. No. 4,622,259 to McAmish et al., each of which is herein incorporated by reference in its entirety. General spunbonding processes are described, for example, in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, and 30 U.S. Pat. No. 3,542,615 to Dobo et al., which are all incorporated herein by reference.

The arrangement and/or configuration of fibers used in the fleece materials can vary, and include fibers having any type of cross-section, including, but not limited to, circular, rectangular, square, oval, triangular, and multilobal. In some embodiments, the fibers can have one or more void spaces, wherein the void spaces can have, for example, circular, rectangular, square, oval, triangular, or multilobal cross-sections. As noted previously, the fibers can be selected from single-component (i.e., uniform in composition throughout the fiber) or multicomponent fiber types including, but not limited to, fibers having a sheath/core structure and fibers having an islands-in-the-sea structure, as well as fibers having a side-by-side, segmented pie, segmented cross, segmented ribbon, or tipped multilobal cross-sections.

The fleece materials described herein can have varying thicknesses, porosities and other parameters. The fleece material can be formed such that the fiber orientation and porosity of the pouched product formed therefrom can retain the composition adapted for oral use that is enclosed within the outer water-permeable pouch, but can also allow the flavors of the composition to be enjoyed by the consumer. For example, in some embodiments, the fleece material can have a basis weight of about 20 gsm to about 35 gsm, and in some such embodiments about 25 gsm to about 30 gsm. In certain embodiments, the fleece material can have a basis weight of about 28 gsm. In some embodiments, the fleece material can have a relatively high basis weight. For example, the basis weight of a fleece material can be in the range of about 25-40 gsm, about 30-40 gsm, or about 35-40 gsm. In certain embodiments, the basis weight of the fleece material can be about 25 gsm or greater, about 30 gsm or greater, or about 35 gsm or greater. Basis weight of a fabric can be measured using ASTM D3776/D3776M-09a (2013) (Standard Test Methods for Mass Per Unit Area (Weight) of Fabric), for example.

In various embodiments, the fleece material can have a thickness of about 0.1 mm to about 0.15 mm (e.g., about 0.11 mm). The fleece material can have an elongation of about 70% to about 80%, e.g., about 78%. In some embodiments, the fleece material can have a peak load of about 4 lbs. to about 8 lbs., e.g., about 5.5 lbs. Elongation and breaking strength of textile fabrics can be measured using ASTM D5034-09(2013) (Standard Test Method for Breaking Strength and Elongation of Textile Fabrics (Grab Test)), for example. In various embodiments, the fleece material can have a Tensile Energy Absorption (TEA) of about 35 to about 40, e.g., about 37. In certain embodiments, the fleece material can have a porosity of greater than about 10,000 ml/min/cm². TEA can be measured, for example, as the work done to break the specimen under tensile loading per lateral area of the specimen. Porosity, or air permeability of textile fabrics can be measured using ASTM D737-04(2012) (Standard Test method for Air Permeability of Textile Fabrics), for example.

### RF Sealable Materials

As noted above, the fleece materials described herein advantageously further comprise a radio frequency (RF) sealable material. “RF sealable materials” as referred to herein may include any polar polymer material (typically a thermoplastic and/or polymer type material) that is capable of bonding together other components of the fleece materials upon application of radio frequency energy (e.g., radio frequency electromagnetic waves and/or electrical currents, as described in further detail herein below). A “polar polymer material” as used herein, refers to any polymer material having a polar molecular structure, e.g., a molecular structure having polar bonds forming dipoles (e.g., a positively charged end and an opposing negatively charged end), wherein the sum of all the bond's electric dipole moments is not equal to zero. Without intending to be bound by theory, it should be noted that when polar molecules within the RF sealable material are exposed to an alternating electric field, for example, they tend to align in the field direction so that the positive end of the dipole will align with the negative charges according to the electric field. When the dipoles reorient according to the high-frequency alternating electric field, their orientation becomes out-of-phase such that misalignment between the dipoles happens and as a result creates internal molecular frictional heating. This internal molecular frictional heating causes the RF sealable material to melt, thereby fusing the RF sealable material with other components of the fleece material to form a seam as will be discussed further herein.

Example RF sealable materials for use with RF sealing techniques according to the present disclosure include, but are not limited to: acrylonitrile butadiene styrene (ABS) resins or polymers, acrylonitrile-methyl acrylate copolymer (AMAC), butyrate, cellulose acetate, cellulose acetate butyrate, cellulose nitrate, cellulose triacetate, various epoxy resins, ethylene-vinyl acetate (EVA), ethyl vinyl alcohol (EVOH), melamine-formaldehyde resin, methyl acrylate, pelathane, polyethylene terephthalate (PET), polyethylene terephthalate glycol-modified (PET-G), polyvinyl acetate (PVA), polyvinylchloride (PVC), polyvinylidene chloride, polyurethane, polyolefin, nylon, thermoplastic polyurethanes, open celled polyurethanes, low-density polyethylene (LDPE), and combinations thereof. Examples of various RF sealable materials suitable for use in various industrial application are described in detail, for example, in U.S. Pat. No. 6,855,778 to Yanuzzi et al.; U.S. Pat. No. 7,220,950 to Gruenspecht et al.; U.S. Pat. No. 7,586,071 to Gruenspecht et al.; and U.S. Pat. No. 9,505,168 to Hinterseer, the disclosures of which are incorporated herein by reference in their entirety.

RF sealable materials as disclosed herein may be incorporated into the fleece material in various different forms and using a variety of different methods. For example, the RF sealable material may be in the form of a plurality of RF sealable fibers, a liquid coating, a powder, a spray coating, and the like. Generally, the RF sealable material (irrespective of form) is applied to, coated on, or combined with, one or more other types of fibers (e.g., such as those described herein above, i.e., non-RF-sealable fibers) prior to, or during, formation of the fleece material. For example, the fleece materials may comprise both a plurality of fibers and an RF sealable material in various forms. In some embodiments, the RF sealable material may, itself, be in the form of a plurality of RF sealable fibers, e.g., such as thermoplastic polymer fibers. In such embodiments, the plurality of RF sealable fibers may be blended with the plurality of fibers in the fleece material (e.g., cellulose fibers, regenerated cellulose fibers, etc.) prior to and/or during formation of the fleece materials as described herein above. In some embodiments, the RF sealable material may be coated onto (e.g., when the RF sealable material is in the form of a liquid or spray coating), or otherwise associated with (e.g., when the RF sealable material is in the form of a powder), the plurality of fibers prior to and/or during formation of the fleece material. In some embodiments, the RF sealable material may additionally, or alternatively, be added directly to fleece materials after formation of the fleece material, e.g., such as in the form of a surface coating layer. In some embodiments, the RF sealable material may be applied to the entire fleece material or to only a portion of the fleece material.

The amount of RF sealable material within the fleece material may vary, for example, based on the particular RF sealable material used, the type of fleece material, the particular frequency of radio waves applied to the fleece material, and the like. Generally, the RF sealable material can be used in an amount sufficient to ensure a permanent seal between one or more layers of fleece material. In some embodiments, the amount of RF sealable material incorporated into the fleece material may be in the range of about 5 percent by weight to about 50 percent by weight, based on the total weight of the fleece material. In some embodiments, the amount of RF sealable material incorporated into the fleece material may be at least about 1 percent by weight of fleece material, at least about 5 percent by weight of the fleece material, at least about 10 percent by weight of the fleece material, at least about 15 percent by weight of the fleece material, or at least about 20 percent by weight of the fleece material.

### RF Sealing Processes and Methods

As noted above, some aspects of the disclosure are directed to methods of RF sealing pouch materials and pouched products formed therefrom. In some embodiments, for example, one or more fleece materials comprising an RF sealable material may be at least partially bonded together using radio frequency (RF) welding/sealing techniques, also commonly referred to as high frequency welding and/or dielectric welding, to form an RF sealed pouch material.

The terms “bond,” “bonded,” “bonding,” “seal,” “sealed,” “sealing,” “weld,” “welded,” “welding,” “fuse,” “fused,” and “fusing” may be used throughout this disclosure with reference to a physical or chemical bond created between one or more fleece materials and generally such terms are meant to be interchangeable as used herein.

Reference to “RF sealing” and/or “RF welding” as used herein, refers to any method of bonding one or more fleece materials together using radio frequency energy to melt a RF sealable material therein, forming one or more seams. For example, application of radio frequency energy (e.g., about 1 MHz to about 100 MHz) to a fleece material comprising a RF sealable material as described herein can fuse the fleece materials together to provide a sealed seam with good strength properties. Generally, “Radio frequency energy” is defined as electromagnetic energy waves having a frequency in the range of about 30 Hz to about 300 GHz; however, the range of radio frequency energy used in the RF sealing processes of the present disclosure is typically between about 1 MHz to about 100 MHz. In some embodiments, the range of radio frequencies that can be used in the RF sealing process is about 1 MHz to about 100 MHz, about 10 MHz to about 70 MHz, or about 20 MHz to about 40 MHz. In some embodiments, the radio frequency energy used in the RF sealing process is at least about 1 MHz, at least about 10 MHz, at least about 20 MHz, at least about 40 MHz, or at least about 60 MHz.

It should be noted that the terms “RF sealing” and “heat sealing” are often (incorrectly) used interchangeably in the industry; however, these methods are distinguishable, as RF sealing generally does not require any external heat source to heat and/or bond the fleece materials as would typically be required with heat sealing processes commonly used for sealing pouched products. This is due to the fact that substantially all of the heat generated in the RF sealing process is due to molecular interactions caused when the radio frequency electromagnetic energy is applied to a material (here, the fleece material). Without intending to be bound by theory, it should be noted that fleece materials that have been sealed using RF sealing processes that do not require an external heat source generally can provide the advantages of more uniform bonding, smaller overall seam widths, and higher bonding strength when compared to fleece materials that have been sealed using conventional heat sealing processes and methods. Various methods and apparatuses generally useful for RF welding/sealing are described in detail in U.S. Pat. No. 5,833,915 to Shah; U.S. Pat. No. 7,220,950 to Gruenspecht et al.; U.S. Pat. No. 7,875,680 to Chen; and U.S. Pat. No. 9,505,168 to Hinterseer, the disclosures of which are incorporated herein by reference in their entireties.

In some embodiments, RF sealing processes as described herein can be conducted using systems comprising two main elements, e.g., an RF generator (e.g., that generates the radio frequency energy) and an RF sealing system (e.g., a mechanical press or die apparatus that compresses layers of fleece materials while the RF energy generated by the RF generator is being applied). Generally, an RF sealing system comprises two electrodes referred to as the RF sealing dies, which are designed to emit radio frequency energy during operation such that this radio frequency energy is transferred to a fleece material. Suitable RF sealing dies may be manufactured using various types of metals, for example, brass or aluminum. It should be noted that the type of material used for the RF sealing die may vary as some type of materials may consume radio frequency energy more quickly. In some embodiments, the mechanical press or die apparatus may additionally utilize an air cylinder or hydraulic press in order to apply mechanical pressure to the fleece material while simultaneously applying radio frequency energy transferred to the RF sealing dies from the RF generator. In such embodiments, the RF sealable material in the fleece material essentially fuses the fleece material together, forming a sealed seam. For example, the electric energy produced via the radio frequency energy causes polar molecules within the RF sealable material to start moving, and this movement generates heat which causes the RF sealable material to soften and thereby fuse adjacent layers together.

Without intending to be bound by theory, RF sealing generally relies on vibration and orientation of charged polar molecules within the polymer chain to generate heat, for example, the movement of these charged molecules releases energy in heat form and when enough energy is applied, the molecules begin to melt and bond. It should be noted then that no outside heat is generally applied and, instead, the heat is generated electromagnetically within the material. In some embodiments, a rapidly alternating electric field is set up between two metal welding bars and the electric field causes the polar molecules found in the fleece materials (e.g., typically associated with the RF sealable materials disclosed herein above) to oscillate and orient themselves with respect to the electric field. The energy generated by this particular process causes a temperature increase which results in melting of the materials.

In some embodiments, the RF welding process may further comprise applying pressure to the fleece materials by clamping the welding bars, further increasing the bond strength. This pressure can be applied before application of the radio frequency energy, simultaneously therewith, and/or after application of the radio frequency energy to complete the weld. Generally, it should be noted that application of pressure is necessary to ensure a uniform RF seal throughout the fleece material. In some embodiments, the amount of pressure applied during the RF sealing process may be between about 20 lbs-per-square-inch (psi) to about 200 psi, about 40 psi to about 160 psi, about 60 psi to about 120 psi, or about 80 psi to about 100 psi. In some embodiments, the pressure applied during the RF sealing process may be at least about 20 psi, at least about 60 psi, at least about 100 psi, or at least about 140 psi.

In some embodiments, a further cooling step may occur during or after application of pressure to the welding site. For example, it should be noted that after cooling the welded surface under maintained pressure, the RF sealable material advantageously becomes fully fused and a strong weld has been created between adjacent layers of material (e.g., creates a permanent seal between one or more fleece materials capable of maintaining the integrity of the pouch product having a composition contained therein). In some embodiments, the cooling time during the RF sealing process may be in the range of about 1 second to about 1 hour, about 5 seconds to about 30 minutes, or about 10 seconds to about 1 minute. In some embodiments, the cooling time during the RF sealing process may be less than about 15 minutes, less than about 10 minutes, less than about 5 minutes, less than about 1 minute, less than about 30 seconds, less than about 15 seconds, or less than about 10 seconds.

In some embodiments, the RF sealing processes described herein may consist of a single cycle or multiple cycles, depending e.g., on the desired weld strength, thickness of the materials, and various other factors as noted herein. In some embodiments, the RF sealing process may comprise at least one cycle, at least two cycles, at least three cycles, at least four cycles, or more. The time required for each cycle may vary and generally will be long enough to ensure a permanent seal has been formed between the fleece materials. In some embodiments, each cycle may consist of multiple stages. For example, in some embodiments, a single cycle may consist of a pre-seal step (e.g., including application of pressure to the fleece material without application of radio frequency energy), followed by a RF sealing step (e.g., including application of radio frequency energy and, optionally, simultaneous application of pressure), followed by a cooling step (e.g., for a certain cooling time and, optionally, with further application of pressure), and finally a cutting step (e.g., including cutting proximate to and/or adjacent to the sealed seams using a die apparatus to remove any excess fleece material and/or to separate individual pouch portions). However, other configurations and/or additional process steps are possible.

In some embodiments, a buffer material may be applied to the RF sealing die in or to prevent heat loss through the RF sealing die surface. Such buffer materials may function as an insulating material by providing a thin layer between the RF sealing die and the fleece material. Generally, addition of a buffer material to the surface of the RF sealing dies can prevent excess heat loss from the die surface, reduce the negative effects of small dents in the die surface, and provide a better seal without arcing. Examples of suitable buffer materials may include, but are not limited to mylar, polyoxybenzylmethylenglycolanhydride (also referred to as Bakelite), polytetrafluoroethylene (also referred to as Teflon), silicone fiberglass, and glassine.

Various types of equipment, components, and apparatuses may be suitable for use in the RF sealing processes described herein. For example, a variety of commercially available RF sealing/welding equipment exists for applications in various other industries, e.g., medical devices, medical bags and waste collection bags, tent and tarp materials, automotive carpets and mats, pool liners and covers, inflatable items (e.g., such as airbags), recovery floats, life vests, seat cushions, and the like. Specific examples of RF sealing apparatuses and components are commercially available from Thermex-Thermatron® Systems, LLC and ONEX RF® Inc. In some embodiments, RF sealing apparatuses used according to the present disclosure may provide for printing on the surface of the fleece material or on the seam of the fleece material. In such embodiments, for example, various branding and/or consumer identifiable information may be printed or stamped on the fleece materials and/or the sealed seams of those fleece materials during the RF welding process.

In some embodiments, the RF sealing processes and systems according to the present disclosure may be controlled using various process control systems. For example, RF welding process control systems are commercially available from ONEX RF® Inc. Example process control systems may control a variety of process parameters, including for example, but not limited to, the pre-seal time, the pre-seal power, the main-seal time, the main-seal power, the cool-time and the like. Such control systems may use various types of programmable control logic (PLC) or programmable controllers (PC) typical known in various industrial processes.

Various machine parameters and/or conditions can affect the RF welding process and the quality of the welds formed therefrom (e.g., the size and/or the strength of those welds forming the seams of RF sealed pouched products). For example, the quality of welds formed by the RF welding processes disclosed herein may be altered based on the combination of machine parameters (e.g., power output or electromagnetic frequency), the temperature profile, the amount of pressure applied, welding efficiency, welding time, cooling time, the type of fleece material, the type of coating and/or binder material applied, the type of RF sealable material used, the thickness of the fleece materials, and combinations thereof. Strength of a RF weld is generally measured by performing a pull test to assess the failure load of the weld, or by examining the weld bead created between the layers of welded material. A “Pull test” may be performed, for example, on a Nonwoven Fabric Pull Tear Tester using Standard FZ/T60005-91 (Non-woven fabric breaking strength and elongation at break measurement).

### Methods of Preparing Pouched Products

As referenced above, some aspects of the present disclosure relate to methods of preparing RF sealed pouched products. It should be noted that any of the RF sealing processes, components, equipment, and/or parameters described herein above may apply as a sealing technique in the general methods of manufacturing pouched products provided herein below.

In one aspect of the present disclosure, a method of preparing an RF sealed pouched product comprises providing an outer-water permeable pouch comprising an RF sealable material (e.g., such as the RF sealable materials described herein above), the outer water-permeable pouch defining a cavity with a composition situated therein; and sealing one or more seams of the outer water-permeable pouch using radio frequency energy to form an RF sealed pouched product (e.g., using any of the RF sealing techniques described herein above). In some embodiments, a method of preparing an RF sealed pouched product may comprise positioning a composition layer between two or more layers of a fleece material (e.g., comprising a plurality of fibers and a RF sealable material) and sealing the outer perimeter of the two or more fleece layers using radio frequency energy to form an RF sealed pouched product encasing the composition, e.g., wherein the entire outer perimeter of the RF sealed pouched product forms a sealed seam. In such embodiments, the RF sealed pouch material may be cut into various shapes and/or sizes using a die apparatus or the like, e.g., as noted above and as depicted in FIGS. 2A, 2B, 2C, and 2D.

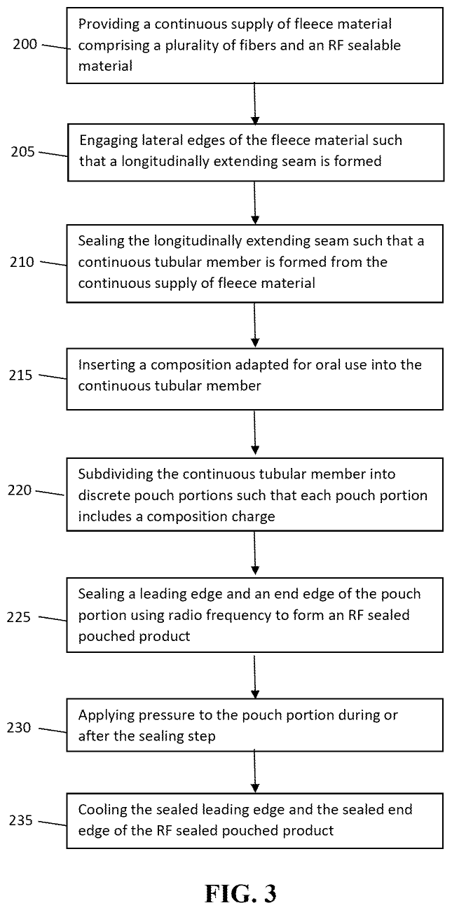

In some embodiments, RF sealed pouch products according to the present disclosure may comprise two seams positioned at opposing ends of the pouched product, e.g., a leading edge and an end edge. Such terms generally refer to a first, front (leading) edge and a second, back (end) edge as defined with respect to the pouched product along a machine direction (e.g., as depicted in FIG. 1). As illustrated in FIG. 3, for example, methods of manufacturing a pouched product can comprise a number of general, non-limiting operations that can be performed in any desirable order prior to the RF sealing process. At operation 200, a continuous supply of fleece material comprising a plurality of fibers and an RF sealable material (e.g., in the form of a plurality of RF sealable fibers blended with the plurality of fibers; or in the form of a liquid coating, a spray coating, and/or a powder incorporated within the fleece material) can be provided. At operation 205, lateral edges of the fleece material are engaged such that a longitudinally extending seam is formed. At operation 210, the fleece material is formed into a continuous tubular member by sealing the lateral edges of the fleece material. This longitudinally-extending seam can be formed by applying conventional heat sealing techniques to the pouch material or any other suitable sealing method generally known in the art. In some embodiments, the longitudinally-extending seam can be formed by applying any of the RF sealing techniques as discussed herein above. At operation 215, a charge of a composition adapted for oral use can be inserted into the continuous tubular member. At operation 220, the continuous tubular member can be subdivided at predetermined intervals so as to form a plurality of outer water-permeable pouch member portions, wherein each pouch member portion includes a charge of the composition. At operation 225, each discrete pouch portion can be entirely sealed such that an outer water-permeable pouch is formed that encloses the composition (e.g., an RF sealed pouched product). This second sealing step can involve applying any of the RF sealing processes and methods described herein above. For example, such methods may comprise sealing a leading edge and/or an end edge (preferably both) of an outer water-permeable pouch using radio frequency energy to form the RF sealed pouched product. As noted above, any of the RF sealing processes and RF sealable materials disclosed herein above may be suitable for use in this particular step. For example, as noted at operation 230, in some embodiments the method may further comprise applying pressure to the outer water-permeable pouch during or after the sealing step using the various parameters noted herein above. Additionally, as noted at operation 235, the sealed leading edge and the sealed end edge of the RF sealed pouched product may be cooled after the sealing step using the various parameters noted herein above. The operations described and the order of the method steps illustrated herein are not construed as limiting thereof.

Various manufacturing apparatuses and methods, in addition to those used with the RF sealing processes and methods described above, can be used to create an RF pouched product as described herein. For example, US Appl. Pub. No. 2012/0055493 to Novak, III et al., previously incorporated by reference in its entirety, relates to an apparatus and process for providing pouch material formed into a tube for use in the manufacture of smokeless tobacco products. Similar apparatuses that incorporate equipment for supplying a continuous supply of a pouch material (e.g., a pouch processing unit adapted to supply a pouch material to a continuous tube forming unit for forming a continuous tubular member from the pouch material) can be used to create a pouched product described herein, wherein the pouch material is a needle-punched fleece as provided herein. Representative equipment for forming such a continuous tube of pouch material is disclosed, for example, in US Appl. Pub. No. 2010/0101588 to Boldrini et al., which is incorporated herein by reference in its entirety. The apparatus further includes equipment for supplying pouched material to the continuous tubular member such that, when the continuous tubular member is subdivided and sealed into discrete pouch portions, each pouch portion includes a charge of a composition adapted for oral use. Representative equipment for supplying the filler material is disclosed, for example, in US Pat. Appl. Pub. No. US 2010/0018539 to Brinkley, which is incorporated herein by reference in its entirety. In some instances, the apparatus may include a subdividing unit for subdividing the continuous tubular member into individual pouch portions and, once subdivided into the individual pouch portions, may also include an RF sealing unit (as described above) for sealing one or more ends of each pouch portion. In other instances, the continuous tubular member may be sealed into individual pouch portions with an RF sealing unit and then, once the individual pouch portions are RF sealed, the continuous tubular member may be subdivided into discrete individual pouch portions by a subdividing unit subdividing the continuous tubular member between the RF sealed ends of serially-disposed pouch portions. Still in other instances, RF sealing (closing) of the individual pouch portions of the continuous tubular member may occur substantially concurrently with the subdivision thereof, using a closing and dividing unit in combination with a typical RF sealing unit.

An example apparatus for manufacturing an oral pouch product is illustrated in FIGS. 1-5 of US Pat. Appl. Pub. No. 2012/0055493 to Novak, III et al.; however, this apparatus is used in a generic and descriptive sense only and not for purposes of limitation. It should also be appreciated that the manufacturing process and related equipment used therein is not limited to the process order described therein and such processes are simply provided to illustrate the types of manufacturing processes and apparatuses that can be used in combination with, generally prior to, application of the RF sealing processes described herein above. In various embodiments of the present disclosure, for example, an apparatus similar to that described in US Pat. Appl. Pub. No. 2012/0055493 can be used in combination with the RF sealing processes and methods described herein to prepare RF sealed pouched products.

The amount of material contained within each RF sealed pouch may vary. In smaller embodiments, the dry weight of the material within each pouch is at least about 50 mg to about 150 mg. For a larger embodiment, the dry weight of the material within each pouch preferably does not exceed about 300 mg to about 500 mg. In some embodiments, the dry weight of the material within each pouch is at least about 50 mg, for example, from about 50 mg to about 2 grams, from about 100 mg to about 1.5 grams, or from about 200 to about 700 mg. In some embodiments, each pouch/container may have disposed therein a flavor agent member, as described in greater detail in U.S. Pat. No. 7,861,728 to Holton, Jr. et al., which is incorporated herein by reference. For example, at least one flavored strip, piece or sheet of flavored water dispersible or water soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and U.S. Pat. No. 6,923,981 to Leung et al.; and The EFSA Journal (2004) 85, 1-32; which are incorporated herein by reference.

In various embodiments, the fleece materials used within the RF sealed pouch materials described herein can be sufficiently tacky so as to create issues with high-speed pouching equipment. Therefore, in certain embodiments, a Teflon® coating, or similar material, can be applied to one or more surfaces of the pouching equipment that touch the fleece material such as, for example, rollers, cutting instruments, and RF sealing devices (as noted above) in order to reduce and/or alleviate any problems associated with the pouch material sticking to the pouching equipment during processing.

The pouched products can further include product identifying information printed or dyed on the outer water-permeable pouch or imprinted (e.g., embossed, debossed, or otherwise pressed) on the outer water-permeable pouch, such as described in US Pat. Appl. Pub. No. 2014/0255452 to Reddick et al., which is incorporated by reference herein. As noted above, flavorants can also be incorporated into the nonwoven web if desired, such as by coating or printing an edible flavorant ink onto the nonwoven web. See, e.g., US Pat. Appl. Pub. Nos. 2012/0085360 to Kawata et al. and 2012/0103353 to Sebastian et al., each of which is herein incorporated by reference.

Products of the present disclosure configured for oral use may be packaged and stored in any suitable packaging in much the same manner that conventional types of smokeless tobacco products are packaged and stored. For example, a plurality of packets or pouches may be contained in a cylindrical container. The storage period of the product after preparation may vary. As used herein, “storage period” refers to the period of time after the preparation of the disclosed product. In some embodiments, one or more of the characteristics of the products disclosed herein (e.g., retention of whiteness, lack of color change, retention of volatile flavor components) is exhibited over some or all of the storage period. In some embodiments, the storage period (i.e., the time period after preparation) is at least one day. In some embodiments, the storage period is from about about 1 day, about 2 days, or about 3 days, to about 1 week, or from about 1 week to about 2 weeks, from about 2 weeks to about 1 month, from about 1 month to about 2 months, from about 2 months to about 3 months, from about 3 months to about 4 months, or from about 4 months to about 5 months. In some embodiments, the storage period is any number of days between about 1 and about 150. In certain embodiments, the storage period may be longer than 5 months, for example, about 6 months, about 7 months, about 8 months, about 9 months, about 10 months, about 11 months, or about 12 months.

### Composition Within the Pouch

Pouched products as described herein and compositions thereof can include various other components, e.g., associated within the composition. The composition of the material within the RF sealed pouched products provided herein is not particularly limited, and can comprise any filling composition, including those included within conventional pouched products. Such compositions are generally mixtures of two or more components and as such, the compositions are, in some cases, referenced herein below as “mixtures.” Such components are not intended to be limiting; rather, various example compositions and components thereof that may be incorporated within pouched products are provided herein below. It is noted that the particular components below are described with specific reference to inclusion within the composition situated within the cavity of the outer water-permeable pouch; however, in various embodiments one or more of such components (e.g., an active ingredient, a flavoring agent, etc.) may be incorporated into the fleece materials as well.

### Active Ingredient

The composition as disclosed herein includes one or more active ingredients. As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, N.Y., 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition;

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-tranlational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Nicotine Component

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the composition, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

### Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and cannabidiol (CBD) another major constituent of the plant, but which is devoid of psychoactivity. All of the above compounds can be used in the form of an isolate from plant material or synthetically derived.

Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids.

When present, a cannabinoid (e.g., CBD) or cannabimimetic is typically in a concentration of at least about 0.1% by weight of the composition, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the composition.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Flavoring Agent

In some embodiments, the composition within the pouch may comprise one or more flavoring agents. As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. In some embodiments, the releasable component may include a single flavoring agent or a plurality of flavoring agents. If desired, one or more flavoring agents may be embedded within the fleece material, absorbed in or adsorbed on at least one surface of the fleece material, or contained within the bulk of the fleece material.

Non-limiting examples of flavoring agents include vanilla, coffee, chocolate/cocoa, cream, mint, spearmint, menthol, peppermint, wintergreen, eucalyptus, lavender, cardamon, nutmeg, cinnamon, clove, cascarilla, sandalwood, honey, jasmine, ginger, anise, sage, licorice, lemon, orange, apple, peach, lime, cherry, strawberry, terpenes, trigeminal senstates, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. Appl. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent may be a volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin.

Any flavoring agent as described herein above is meant to be suitable for use within the composition within the pouch. The amount of flavoring agent utilized in the composition can vary, but is typically up to about 10 weight percent, and certain embodiments are characterized by a flavoring agent content of at least about 0.1 weight percent, such as about 0.5 to about 10 weight percent, about 1 to about 6 weight percent, or about 2 to about 5 weight percent, based on the total weight of the composition.

### Filler Component

In some embodiments, the composition may include at least one particulate filler component. Such particulate filler components may fulfill multiple functions, such as enhancing certain organoleptic properties such as texture and mouthfeel, enhancing cohesiveness or compressibility of the product, and the like. Generally, the filler components are porous particulate materials and are cellulose-based. For example, suitable particulate filler components are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential particulate filler components include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated di starch adipate, acetylated di starch glycerol, hydroxypropyl starch, hydroxypropyl distarch glycerol, starch sodium octenyl succinate.

In some embodiments, the particulate filler component is a cellulose material or cellulose derivative. One particularly suitable particulate filler component for use in the products described herein is microcrystalline cellulose (“MCC”). The MCC may be synthetic or semi-synthetic, or it may be obtained entirely from natural celluloses. The MCC may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In one embodiment, the mixture comprises MCC as the particulate filler component. The quantity of MCC present in the mixture as described herein may vary according to the desired properties.

The amount of particulate filler component can vary, but is typically up to about 75 percent of the composition by weight, based on the total weight of the composition. A typical range of particulate filler material (e.g., MCC) within the composition can be from about 10 to about 75 percent by total weight of the composition, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of particulate filler material is at least about 10 percent by weight, such as at least about 20 percent, or at least about 25 percent, or at least about 30 percent, or at least about 35 percent, or at least about 40 percent, based on the total weight of the composition.

In one embodiment, the particulate filler component further comprises a cellulose derivative or a combination of such derivatives. In some embodiments, the mixture comprises from about 1 to about 10% of the cellulose derivative by weight, based on the total weight of the mixture, with certain embodiments comprising about 1 to about 5% by weight of cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. In some embodiments, the mixture comprises from about 1 to about 3% HPC by weight, based on the total weight of the mixture.

### Tobacco Material

In some embodiments, the composition may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Pat. Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; U.S. Pat. No. 5,387,416 to White et al.; and U.S. Pat. No. 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The composition disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the composition most preferably includes tobacco lamina, or a tobacco lamina and stem mixture (of which at least a portion is smoke-treated). Portions of the tobaccos within the composition may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Pat. No. 7,556,047 to Poindexter, et al., all of which are incorporated by reference. In addition, the composition optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

For the preparation of oral products, it is typical for a harvested plant of the Nicotiana species to be subjected to a curing process. The tobacco materials incorporated within the composition for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int., 21, 305-320 (2005) and Staaf et al., Beitrage Tabakforsch. Int., 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496; 8,944,072; and U.S. Pat. No. 8,991,403 to Chen et al., which are all incorporated herein by reference. In certain embodiments, this type of treatment is useful where the original tobacco material is subjected to heat in the processes previously described.

In some embodiments, the type of tobacco material is selected such that it is initially visually lighter in color than other tobacco materials to some degree (e.g., whitened or bleached). Tobacco pulp can be whitened in certain embodiments according to any means known in the art. For example, bleached tobacco material produced by various whitening methods using various bleaching or oxidizing agents and oxidation catalysts can be used. Example oxidizing agents include peroxides (e.g., hydrogen peroxide), chlorite salts, chlorate salts, perchlorate salts, hypochlorite salts, ozone, ammonia, potassium permanganate, and combinations thereof. Example oxidation catalysts are titanium dioxide, manganese dioxide, and combinations thereof. Processes for treating tobacco with bleaching agents are discussed, for example, in U.S. Pat. Nos. 787,611 to Daniels, Jr.; U.S. Pat. No. 1,086,306 to Oelenheinz; U.S. Pat. No. 1,437,095 to Delling; U.S. Pat. No. 1,757,477 to Rosenhoch; U.S. Pat. No. 2,122,421 to Hawkinson; U.S. Pat. No. 2,148,147 to Baier; U.S. Pat. No. 2,170,107 to Baier; U.S. Pat. No. 2,274,649 to Baier; U.S. Pat. No. 2,770,239 to Prats et al.; U.S. Pat. No. 3,612,065 to Rosen; U.S. Pat. No. 3,851,653 to Rosen; U.S. Pat. No. 3,889,689 to Rosen; U.S. Pat. No. 3,943,940 to Minami; U.S. Pat. No. 3,943,945 to Rosen; U.S. Pat. No. 4,143,666 to Rainer; U.S. Pat. No. 4,194,514 to Campbell; U.S. Pat. Nos. 4,366,823, 4,366,824, and U.S. Pat. No. 4,388,933 to Rainer et al.; U.S. Pat. No. 4,641,667 to Schmekel et al.; U.S. Pat. No. 5,713,376 to Berger; U.S. Pat. No. 9,339,058 to Byrd Jr. et al.; U.S. Pat. No. 9,420,825 to Beeson et al.; and U.S. Pat. No. 9,950,858 to Byrd Jr. et al.; as well as in US Pat. Appl. Pub. Nos. 2012/0067361 to Bjorkholm et al.; 2016/0073686 to Crooks; 2017/0020183 to Bjorkholm; and 2017/0112183 to Bjorkholm, and in Int. Appl. Pub. Nos. WO1996/031255 to Giolvas and WO2018/083114 to Bjorkholm, all of which are incorporated herein by reference.

In some embodiments, the whitened tobacco material can have an ISO brightness of at least about 50%, at least about 60%, at least about 65%, at least about 70%, at least about 75%, or at least about 80%. In some embodiments, the whitened tobacco material can have an ISO brightness in the range of about 50% to about 90%, about 55% to about 75%, or about 60% to about 70%. ISO brightness can be measured according to ISO 3688:1999 or ISO 2470-1:2016.

In some embodiments, the whitened tobacco material can be characterized as lightened in color (e.g., “whitened”) in comparison to an untreated tobacco material. White colors are often defined with reference to the International Commission on Illumination's (CIE's) chromaticity diagram. The whitened tobacco material can, in certain embodiments, be characterized as closer on the chromaticity diagram to pure white than an untreated tobacco material.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; U.S. Pat. No. 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; U.S. Pat. No. 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and U.S. Pat. No. 7,337,782 to Thompson, all of which are incorporated by reference herein.

Typical inclusion ranges for tobacco materials can vary depending on the nature and type of the tobacco material, and the intended effect on the final mixture, with an example range of up to about 30% by weight (or up to about 20% by weight or up to about 10% by weight or up to about 5% by weight), based on total weight of the mixture (e.g., about 0.1 to about 15% by weight).

It should be noted that inclusion of a tobacco material into the compositions and products described herein is meant to be optional and is not required. In some embodiments, oral products as described herein can generally be characterized as being tobacco free-alternatives. For example, in some embodiments, oral products of the present disclosure may be said to be completely free or substantially free of tobacco material (other than purified nicotine as an active ingredient). Oral products that are referred to as “completely free” of or “substantially free of” a tobacco material herein are meant to refer to oral products that can be characterized as having less than about 1.0% by weight, less than about 0.5% by weight, less than about 0.1% by weight of tobacco material, or 0% by weight of tobacco material.

Further Additives

In some embodiments, one or more further additives can be included in the composition within the pouched products. For example, the compositions can be processed, blended, formulated, combined and/or mixed with other materials or ingredients. The additives can be artificial, or can be obtained or derived from herbal or biological sources. Specific types of further additives that may be included are further described below.

In some embodiments, the composition may include a content of water. The water content of the composition within the product, prior to use by a consumer of the product, may vary according to the desired properties. Typically, the composition, as present within the product prior to insertion into the mouth of the user, can comprise less than 60%, less than 50%, less than 40%, less than 30%, less than 20%, less than 10%, or less than 5% by weight of water. For example, total water content in the composition and/or product may be in the range of about 0.1% to about 60%, about 1% to about 50%, about 1.5% to about 40%, or about 2% to about 25% by weight of water. In some embodiments, the compositions and products may include at least 1%, at least 2%, at least 5%, at least 10%, or at least 20% by weight water.

In some embodiments, the composition may include a content of one or more organic acids. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific ingredient as opposed to merely being inherently present as a component of another ingredient (e.g., the small amount of organic acid which may inherently be present in an ingredient such as a tobacco material). In some embodiments, the one or more organic acids are added neat (i.e., in their free acid, native solid or liquid form) or as a solution in, e.g., water. In some embodiments, the one or more organic acids are added in the form of a salt, as described herein below.

In some embodiments, the organic acid is an alkyl carboxylic acid. Non-limiting examples of alkyl carboxylic acids include formic acid, acetic acid, propionic acid, octanoic acid, nonanoic acid, decanoic acid, undecanoic acid, dodecanoic acid, stearic acid, oleic acid, linoleic acid, linolenic acid, and the like. In some embodiments, the organic acid is an alkyl sulfonic acid. Non-limiting examples of alkyl sulfonic acids include propanesulfonic acid and octanesulfonic acid. In some embodiments, the alkyl carboxylic or sulfonic acid is substituted with one or more hydroxyl groups. Non-limiting examples include glycolic acid, 4-hydroxybutyric acid, and lactic acid. In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like. In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid. In some embodiments, the organic acid is citric acid, malic acid, tartaric acid, octanoic acid, benzoic acid, a toluic acid, salicylic acid, or a combination thereof. In some embodiments, the organic acid is benzoic acid. In some embodiments, the organic acid is citric acid. In alternative embodiments, a portion, or even all, of the organic acid may be added in the form of a salt with an alkaline component, which may include, but is not limited to, nicotine. Non-limiting examples of suitable salts, e.g., for nicotine, include formate, acetate, propionate, isobutyrate, butyrate, alpha-methylbutyate, isovalerate, beta-methylvalerate, caproate, 2-furoate, phenylacetate, heptanoate, octanoate, nonanoate, oxalate, malonate, glycolate, benzoate, tartrate, levulinate, ascorbate, fumarate, citrate, malate, lactate, aspartate, salicylate, tosylate, succinate, pyruvate, and the like.

The amount of organic acid present in the compositions may vary. Generally, the compositions can comprise from 0 to about 10% by weight of organic acid, present as one or more organic acids, based on the total weight of the composition.

In some embodiments, the composition may further comprise a salt (e.g., alkali metal salts), typically employed in an amount sufficient to provide desired sensory attributes to the compositions and products. Non-limiting examples of suitable salts include sodium chloride, potassium chloride, ammonium chloride, flour salt, and the like. When present, a representative amount of salt is about 0.5 percent by weight or more, about 1.0 percent by weight or more, or at about 1.5 percent by weight or more, but will typically make up about 10 percent or less of the total weight of the composition or product, or about 7.5 percent or less or about 5 percent or less (e.g., about 0.5 to about 5 percent by weight).

The composition also may include one or more sweeteners. The sweeteners can be any sweetener or combination of sweeteners, in natural or artificial form, or as a combination of natural and artificial sweeteners. Examples of natural sweeteners include fructose, sucrose, glucose, maltose, mannose, galactose, isomaltulose, lactose, stevia, honey, and the like. Examples of artificial sweeteners include sucralose, maltodextrin, saccharin, aspartame, acesulfame K, neotame and the like.

In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). When present, a representative amount of sweetener may make up from about 0.1 to about 20 percent or more of the of the composition by weight, for example, from about 0.1 to about 1%, from about 1 to about 5%, from about 5 to about 10%, or from about 10 to about 20% of the composition based on the total weight of the composition.

In some embodiments, the composition may include one or more binding agents. A binder (or combination of binders) may be employed in certain embodiments, in amounts sufficient to provide the desired physical attributes and physical integrity to the composition, and binders also often function as thickening or gelling agents. Typical binders can be organic or inorganic, or a combination thereof. Representative binders include povidone, sodium alginate, starch-based binders, pectin, carrageenan, pullulan, zein, and the like, and combinations thereof. In some embodiments, the binder comprises pectin or carrageenan or combinations thereof. The amount of binder utilized can vary, but is typically up to about 30 weight percent, and certain embodiments are characterized by a binder content of at least about 0.1% by weight, such as about 1 to about 30% by weight, or about 5 to about 10% by weight, based on the total weight of the composition.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the composition.

In certain embodiments, one or more humectants may be employed in the compositions. Examples of humectants include, but are not limited to, glycerin, propylene glycol, and the like. Where included, the humectant is typically provided in an amount sufficient to provide desired moisture attributes to the compositions. Further, in some instances, the humectant may impart desirable flow characteristics to the composition for depositing in a mold. When present, a humectant will typically make up about 5% or less of the weight of the composition (e.g., from about 0.5 to about 5% by weight). When present, a representative amount of humectant is about 0.1% to about 1% by weight, or about 1% to about 5% by weight, based on the total weight of the composition.

In certain embodiments, compositions of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the weight of the composition, for example, from about 0.5% to about 5%, such as, e.g., from about 0.75% to about 4%, from about 0.75% to about 3%, or from about 1% to about 2% by weight, based on the total weight of the composition. Non-limiting examples of suitable buffers include alkali metals acetates, glycinates, phosphates, glycerophosphates, citrates, carbonates, hydrogen carbonates, borates, or mixtures thereof.

In some embodiments, the composition may include one or more colorants. A colorant may be employed in amounts sufficient to provide the desired physical attributes to the composition or product. Examples of colorants include various dyes and pigments, such as caramel coloring and titanium dioxide. The amount of colorant utilized in the compositions or products can vary, but when present is typically up to about 3 weight percent, such as from about 0.1%, about 0.5%, or about 1%, to about 3% by weight, based on the total weight of the composition.

Examples of even further types of additives that may be present in the composition include thickening or gelling agents (e.g., fish gelatin), emulsifiers, oral care additives (e.g., thyme oil, eucalyptus oil, and zinc), preservatives (e.g., potassium sorbate and the like), disintegration aids, zinc or magnesium salts selected to be relatively water soluble for compositions with greater water solubility (e.g., magnesium or zinc gluconate) or selected to be relatively water insoluble for compositions with reduced water solubility (e.g. magnesium or zinc oxide), or combinations thereof. See, for example, those representative components, combination of components, relative amounts of those components, and manners and methods for employing those components, set forth in U.S. Pat. No. 9,237,769 to Mua et al. and U.S. Pat. No. 7,861,728 to Holton, Jr. et al.; and in US Pat. Appl. Pub. Nos. 2010/0291245 to Gao et al. and US Pat. Appl. Pub. No. 2007/0062549 to Holton, Jr. et al., each of which is incorporated herein by reference. Typical inclusion ranges for such additional additives can vary depending on the nature and function of the additive and the intended effect on the final mixture, with an example range of up to about 10% by weight, based on total weight of the composition (e.g., about 0.1 to about 5% by weight).

The aforementioned additives can be employed together (e.g., as additive formulations) or separately (e.g., individual additive components can be added at different stages involved in the preparation of the final composition). Furthermore, the aforementioned types of additives may be encapsulated as provided in the final product or composition. Exemplary encapsulated additives are described, for example, in WO2010/132444 to Atchley, which has been previously incorporated by reference herein.

Particles

In some embodiments, any one or more of a filler component, a tobacco material, and the overall composition described herein can be described as a particulate material. As used herein, the term “particulate” refers to a material in the form of a plurality of individual particles, some of which can be in the form of an agglomerate of multiple particles, wherein the particles have an average length to width ratio less than 2:1. In some embodiments, the particles have an average length to width ratio less than 1.5:1, such as about 1:1. In various embodiments, the particles of a particulate material can be described as substantially spherical or granular.

The particle size of a particulate material may be measured by sieve analysis. As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e. the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

In some embodiments, the column of sieves may be placed on or in a mechanical agitator. The agitator causes the vibration of each of the sieves in the column. The mechanical agitator may be activated for a pre-determined period of time in order to ensure that all particles are collected in the correct sieve. In some embodiments, the column of sieves is agitated for a period of time from 0.5 minutes to 10 minutes, such as from 1 minute to 10 minutes, such as from 1 minute to 5 minutes, such as for approximately 3 minutes. Once the agitation of the sieves in the column is complete, the material collected on each sieve is weighed. The weight of each sample on each sieve may then be divided by the total weight in order to obtain a percentage of the mass retained on each sieve. As the skilled person will readily appreciate, the screen opening sizes or mesh sizes for each sieve in the column used for sieve analysis may be selected based on the granularity or known maximum/minimum particle sizes of the sample to be analysed. In some embodiments, a column of sieves may be used for sieve analysis, wherein the column comprises from 2 to 20 sieves, such as from 5 to 15 sieves. In some embodiments, a column of sieves may be used for sieve analysis, wherein the column comprises 10 sieves. In some embodiments, the largest screen opening or mesh sizes of the sieves used for sieve analysis may be 1000 μm, such as 500 μm, such as 400 μm, such as 300 μm.

In some embodiments, any particulate material referenced herein (e.g., filler component, tobacco material, and the overall composition) can be characterized as having at least 50% by weight of particles with a particle size as measured by sieve analysis of no greater than about 1000 μm, such as no greater than about 500 μm, such as no greater than about 400 μm, such as no greater than about 350 μm, such as no greater than about 300 μm. In some embodiments, at least 60% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 70% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 80% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 90% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 95% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 99% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, approximately 100% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm.

In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 0.01 μm to about 1000 μm, such as from about 0.05 μm to about 750 μm, such as from about 0.1 μm to about 500 μm, such as from about 0.25 μm to about 500 μm. In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 10 μm to about 400 μm, such as from about 50 μm to about 350 μm, such as from about 100 μm to about 350 μm, such as from about 200 μm to about 300 μm.

Preparation of the Mixture

The manner by which the various components of the composition are combined may vary. As such, the overall mixture of various components within the composition, e.g., powdered mixture components, may be relatively uniform in nature. The components noted above, which may be in liquid or dry solid form, can be admixed in a pretreatment step prior to mixture with any remaining components of the composition, or simply mixed together with all other liquid or dry ingredients. The various components of the composition may be contacted, combined, or mixed together using any mixing technique or equipment known in the art. Any mixing method that brings the composition ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as Ploughshare® types of mixer cylinders from Littleford Day, Inc., such as FKM130, FKM600, FKM1200, FKM2000 and FKM3000, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. In some embodiments, the components forming the composition are prepared such that the mixture thereof may be used in a starch molding process for forming the mixture. Manners and methods for formulating mixtures will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and U.S. Pat. No. 6,077,524 to Bolder et al., each of which is incorporated herein by reference.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A pouched product comprising:
an outer water-permeable pouch defining a cavity; and
a composition situated in the cavity;
wherein the outer water-permeable pouch comprises a fleece material, the fleece material comprising a plurality of fibers and an RF sealable material.

2. The pouched product of claim 1, wherein the RF sealable material is a polar polymer material.

3. The pouched product of claim 1, wherein the RF sealable material is selected from the group consisting of acrylonitrile butadiene styrene (ABS) resins or polymers, acrylonitrile-methyl acrylate copolymer (AMAC), butyrate, cellulose acetate, cellulose acetate butyrate, cellulose nitrate, cellulose triacetate, various epoxy resins, ethylene-vinyl acetate (EVA), ethyl vinyl alcohol (EVOH), melamine-formaldehyde resin, methyl acrylate, pelathane, polyethylene terephthalate (PET), polyethylene terephthalate glycol-modified (PET-G), polyvinyl acetate (PVA), polyvinylchloride (PVC), polyvinylidene chloride, polyurethane, polyolefin, nylon, thermoplastic polyurethanes, open celled polyurethanes, low-density polyethylene (LDPE), and combinations thereof.

4. The pouched product of claim 1, wherein the RF sealable material is in the form of a plurality of RF sealable fibers, a liquid coating, a spray coating, a powder, or any combination thereof.

5. The pouched product of claim 1, further comprising a sealed seam.

6. The pouched product of claim 5, wherein the sealed seam has been sealed via application of radio frequency energy.

7. The pouched product of claim 6, wherein the sealed seam has a width of less than about 2 mm.

8. The pouched product of claim 1, wherein the composition within the cavity of the pouch comprises a particulate tobacco material, nicotine, a particulate non-tobacco material treated to contain nicotine and/or flavoring agents, fibrous plant material carrying a tobacco extract, and combinations thereof.

9. The pouched product of claim 8, wherein the particulate tobacco material is in the form of a whitened tobacco material.

10. The pouched product of claim 1, wherein the composition is substantially free of a tobacco material.

11. The pouched product of claim 1, wherein the composition comprises an active ingredient selected from the group consisting of a nicotine component, botanicals, stimulants, medicinals, nutraceuticals, amino acids, vitamins, cannabinoids, terpenes, antioxidants and combinations thereof.

12. The pouched product of claim 1, wherein the composition comprises one or more additives selected from the group consisting of a salt, a sweetener, a binding agent, water, a humectant, a gum, an organic acid, a buffering agent, a tobacco derived material, and combinations thereof.

13. A method of RF sealing pouch materials, comprising:
providing one or more fleece materials comprising an RF sealable material;
sealing the one or more fleece materials along a seam using radio frequency energy to form an RF sealed pouch material.

14. The method of claim 13, wherein sealing the one or more fleece materials comprises application of radio frequency energy to the seam in a range of about 1 MHz to about 100 MHz.

15. The method of claim 14, wherein the radio frequency energy is applied via an RF sealing die.

16. The method of claim 15, wherein the RF sealing die is in the form of two or more electrodes configured to emit radio frequency energy.

17. The method of claim 13, further comprising applying pressure to the one or more fleece materials.

18. The method of claim 17, wherein an amount of pressure applied to the one or more fleece materials is in the range of about 20 psi to about 200 psi.

19. The method of claim 17, wherein applying pressure comprises applying pressure via mechanical press, hydraulic press, pneumatic press, or any combination thereof.

20. The method of claim 13, further comprising cooling the pouch material after sealing.

21. The method of claim 13, wherein the RF sealed pouch material comprises a sealed seam.

22. The method of claim 21, wherein the sealed seam has a width of less than about 2 mm.

23. A method of preparing an RF sealed pouched product, the method comprising:
providing an outer-water permeable pouch comprising a plurality of fibers and a RF sealable material, the outer water-permeable pouch defining a cavity with a composition situated therein; and
sealing a seam of the outer water-permeable pouch using radio frequency energy to form an RF sealed pouched product.

24. The method of claim 23, further comprising applying pressure to the seam of the outer water-permeable pouch.

25. The method of claim 23, further comprising cooling the RF sealed pouch product after sealing.

26. The method of claim 23, further comprising:
providing a continuous supply of a fleece material comprising a plurality of fibers and an RF sealable material;
engaging lateral edges of the fleece material such that a longitudinally-extending seam is formed;
sealing the longitudinally-extending seam such that a continuous tubular member is formed from the continuous supply of fleece material;
inserting a composition adapted for oral use into the continuous tubular member; and
subdividing the continuous tubular member into discrete pouch portions such that each pouch portion includes a composition charge.

27. A pouched product prepared according to the method of claim 23.

28. The pouched product of claim 27, wherein the sealed seam has a width of less than about 2 mm.

29. The pouched product of claim 28, wherein the pouched product exhibits enhanced organoleptic properties as compared to a pouched product that has not been sealed using radio frequency energy.

30. The pouched product of claim 29, wherein the enhanced organoleptic properties are selected from the group consisting of texture, mouthfeel, softness, stiffness, firmness, hardness, stickiness, fluffiness, durability, chewability, workability, tackiness, and combinations thereof.
